(19) United States
(12) Patent Application Publication
(10) Pub. No.: US 2008/0086050 A1
(43) Pub. Date: Apr. 10, 2008

(54) MRI HYPERTHERMIA TREATMENT SYSTEMS, METHODS AND DEVICES, ENDORECTAL COIL

(75) Inventors: George J. Misic, Allison Park, PA (US); Alan D. Hirschman, Glenshaw, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 11/868,552

(22) Filed: Oct. 8, 2007

Related U.S. Application Data
(60) Provisional application No. 60/828,726, filed on Oct. 9, 2006.

(51) Int. Cl. A61B 5/05

(52) U.S. Cl. 600/411; 607/100

(57) ABSTRACT

Hyperthermia has been applied by, for example, separate ultrasound transducers, RF or microwave transmitters and heated fluids. Imaging by separate MRI imaging coils is usually used to view the anatomical region under treatment. Separate temperature probes (needles, catheters) are often used to monitor tissue temperature. Control of the temperature profile required for effective hyperthermia treatment is usually done by trial and error, involving a human operator. The present invention combines all of these capabilities into a single device, which is MRI compatible and safe. It also allows for automatic control of the RF energy to achieve a prescribed tissue hyperthermia.

### CROSS REFERENCE TO RELATED APPLICATION

This application claims an invention which was disclosed in Provisional Application No. 60/828,726, filed Oct. 9, 2006, entitled “MRI Hyperthermia Treatment Systems, Methods and Devices, Endorectal Coil”. The benefit under 35 USC §119(e) of the United States provisional application is hereby claimed, and the aforementioned application is hereby incorporated herein by reference.

### FIELD OF THE INVENTION

The invention relates to a set of MR imaging devices and methods which can be used both to generate images during receive/imaging mode and to deliver RF energy to the imaged tissue during transmit/hyperthermia mode in order to perform controlled hyperthermia. It is known that hyperthermia can be a very effective therapy adjunct to radiation and/or chemotherapy in the treatment of tumors. Hyperthermia differs from thermal ablation therapies in that thermal ablation destroys tissue by the application of heat energy.

### BACKGROUND OF THE INVENTION

The systems, methods and devices of the present invention allow the precise delivery of energy to targeted tissue identified in MR images in order to heat that tissue to a temperature between 40-42° C. Temperature contour maps, accurate to +/−0.5° C., can be generated by frequency-shift MRI and superimposed on anatomical images created by the same set of coils. These temperature maps can be used to create a hyperthermia temperature field, which can be manually controlled by an operator or automatically adjusted by the MRI computer. More particularly, treatment of prostate and colon cancer by adjunctive hyperthermia can be accomplished with modification to, for example, endorectal coils provided by MEDRAD, Inc. (“MEDRAD”), such as shown and described in U.S. Pat. Nos. 5,307,814, 5,348,010, 5,355,087, 5,451,232, and 5,476,095, and U.S. Patent Application Publication No. US20040236209A1, System and Method of Obtaining Images and Spectra of Intracavity Structures Using 3.0 Tesla Magnetic Resonance Systems, the contents of which are incorporated herein by reference. Hyperthermia is a valuable cancer treatment used adjunctively with chemotherapy and/or radiation therapy. Heating a well-defined volume of tumor tissue to 40-42° C. for a specific time period, typically about one hour, can significantly enhance the effectiveness of the primary therapies.

Numerous papers have been published on the clinical application of hyperthermia, including the following: [1] M. H. Falk, R. D. Issels, Hyperthermia in Oncology, International Journal of Hyperthermia, 2000, Volume 17, Number 1, pages 1-18, [2] P. Stauffer, Evolving Technology for Thermal Therapy of Cancer, International Journal of Hyperthermia, December 2005, Volume 21, Number 8, pages 731-744, [3] M. Dewhirst, et al, Re-Setting the Biologic Rationale for Thermal Therapy, International Journal of Hyperthermia, December 2005; Volume 21, Number 8, pages 779-790, [4] Chopra, C. Luginbuhl, A. J. Weymouth, F. S. Foster, M. J. Bronskill. Interstitial Ultrasound Heating Applicator for MR-Guided Thermal Therapy. Phys. Med. Biol. 46:1-13, 2001, [5] US patent publication US20030028097A1; Immobilizer Probe System and Method, D'Amico et al, [6] U.S. Pat. No. 6,957,108B2; Invasive Microwave Antenna Array for Hyperthermia and Brachytherapy, assigned to BSD Medical, Inc.

A major commercial supplier of hyperthermia equipment is BSD Medical, Inc., which is currently testing a device (BSD2000/3D/MR) that delivers RF energy to tissue in an MR scanner via external electromagnetic antennae, and also provides thermal maps superimposed on anatomical MR images. Further, US Patent Application Publication US2003-0028097A1 describes the use of an RF transmitter device for delivery of hyperthermia to tissue from within a body cavity.

Thermal mapping of tissue by means of MRI is known in the art. Chopra, et al “Interstitial Ultrasound Heating Applicator for MR-Guided Thermal Therapy”. Phys. Med. Biol. 46:1-13, 2001, describe a system that heats tissue with focused ultrasound energy and superimposes temperature contour maps obtained by frequency-shift MRI upon the anatomical images.

### SUMMARY OF THE INVENTION

The present invention provides, in one aspect, an imaging system and one or more RF antennae that are inserted into a patient's body cavity or applied to the patient's body to transmit RF energy and cause thermal heating into specific tissue under treatment by radiation or chemotherapy. The same RF antennae, or optionally, related antennae, are operated as MR receiver coils to image the tissue anatomy and temperature distribution achieved in the tissue. In particular, the system and transmit/receive antenna arrays induce a moderate rise to the temperature of the prostate gland, cancerous segments of the colon, or other target anatomy; this is expected to be of therapeutic benefit in many cases. Ideally, the device should also be capable of imaging the prostate, colon, or other target using MRI in a manner similar to that achieved by, for example, MEDRAD endorectal coils or other dedicated surface coils, Interface Devices, and endorectal coil systems for prostate imaging referenced in U.S. Pat. Nos. 5,348,010 and 5,355,087, the contents of which are hereby incorporated herein by reference.

The present invention involves the use of RF energy to provide the heating from a device that is possibly similar in construction to MEDRAD prostate endorectal coils, including the BPX-30, BPX-15, and BPX-10 coils. A particular purpose of the present invention is to provide a controlled amount of heating to the prostate gland or segment of colon tissue affected by cancer; the temperature rise believed to be needed is likely to be 10° C. or less, most likely about 4° to 5° C. The device should ideally still provide imaging capability of the prostate in a manner similar to that provided by MEDRAD BPX family of coil products.

The imaging coils of the present invention can be used to create MR frequency-shift temperature maps of tissue in three dimensions to monitor selective heating of tissue structures. These coils could include the current BPX family of endorectal coils provided by MEDRAD, or new coils with added features such as phased array design, dual-frequency tuning to support imaging at the system Larmour frequency and RF heating at a different frequency, or both new features. The temperature maps could be used to adjust the level and phasing of power to various elements of a coil array in order to control or maintain the desired temperature field. The operator could enter a desired temperature pattern in a volume of tissue as part of an initial image-guided treatment plan. Removing the operator from the loop may be accomplished by providing an intelligent computer control system. This computer control system would use anatomical images and superimposed temperature maps to automatically achieve a desired hyperthermia temperature pattern over a tissue volume.

The MR imaging system could be configured to remember the spatial and temporal patterns of heating in a typical patient. This would allow the hyperthermia treatment plan to be repeated consistently from one treatment fraction to another.

The mechanical properties of an inflatable balloon-like structure also holds the tissue structures firmly and positions those structures for consistent imaging. This allows for accurate and repeatable administration of power to tissue over several treatment sessions. This technique has been reported by D'Amico, et al and is embodied in U.S. Patent Application Publication No. US2003-0028097A1, the contents of which are hereby incorporated herein by reference.

It is also possible to selectively apply energy to achieve a temperature pattern by creating “apertures” over portions of the coil. By selectively applying conductive shields over segments of the coil during power transmission, energy can be channeled through apertures to tissue while being blocked by the shields. In operation, the shields would be removed during imaging and then applied by the operator during a separate power transmission period.

The overall concepts described above can be extended beyond the internal MR endorectal coil applications in hyperthermia. It is possible to apply the same combination of receiver and transmitter coils to other parts of the body. For example, in MR image-guided hyperthermia of the liver, pancreas, colon, or other visceral organs, a flexible coil array can be wrapped around the patient's abdomen. It can then be inflated or strapped in place to hold it in position while MR imaging and heating are performed.

Although many of the applications described here involve MR image-guided hyperthermia, the coils can be used independently of MR scanners purely as RF energy transmitters. Temperature information can be obtained without imaging, e.g. using temperature probes or sensing needles inserted into the target tissue.

### BRIEF DESCRIPTION OF THE DRAWINGS

FIG. 1A is a representation of a system of the present invention.

FIG. 1B is a first embodiment of the present invention.

FIG. 1C is a second embodiment of the present invention.

FIG. 2A is an alternate embodiment of the present invention.

FIG. 2B is another alternate embodiment of the invention using a 1.5 Tesla MRI Imaging System

FIG. 2C is yet another alternate embodiment of the invention using a 3.0 Tesla MRI Imaging System

FIG. 3A is a further alternate embodiment of the invention using a dual tuned endorectal coil for imaging and heating.

FIG. 3B is another alternate embodiment of the invention using a dual tuned endorectal coil for imaging and heating.

FIG. 4A is an alternate embodiment of the invention using the host MR system as an RF source for RF heating.

FIG. 4B is a further alternate embodiment of the invention using a phased array single tuned endorectal coil.

FIG. 5A is an alternate embodiment of the invention using the Host MR System to control the selection of heating coil elements.

FIG. 5B is an alternate embodiment of the invention using a Transmit/Receive interface detail for a phased array dual tuned endorectal coil.

### DETAILED DESCRIPTION OF THE EMBODIMENTS OF THE INVENTION

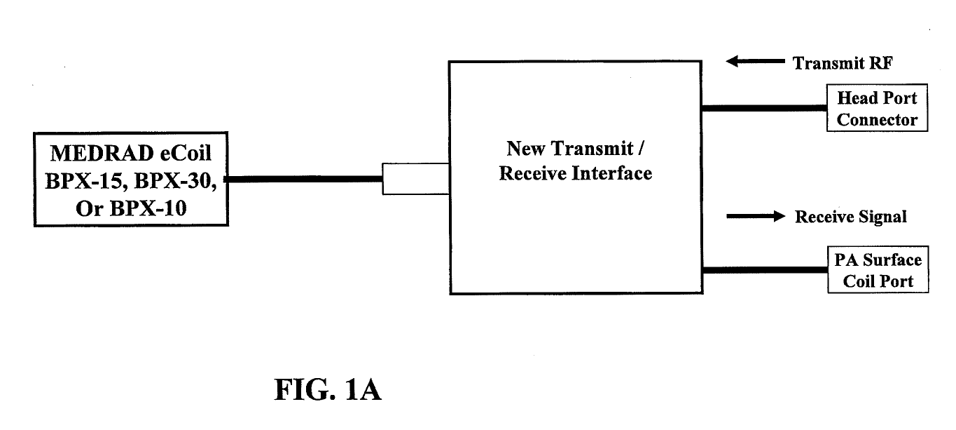

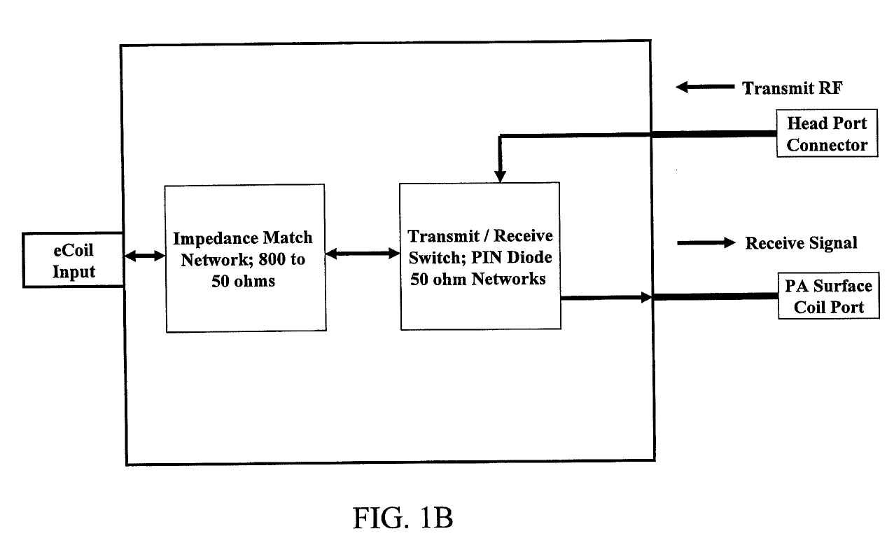

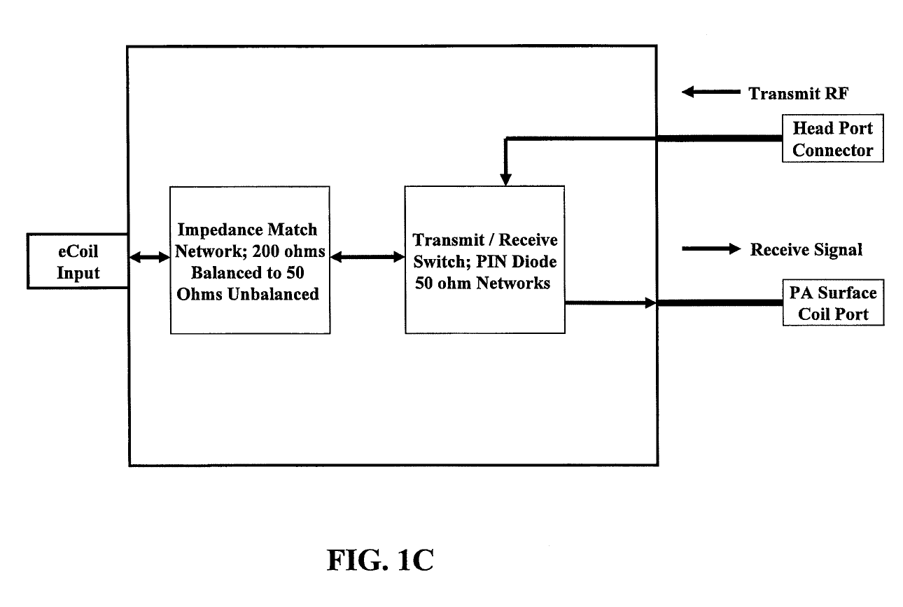

FIGS. 1A, 1B AND 1C uses the existing MEDRAD BPX-10, BPX-15, and BPX-30 as a Transmit/Receive Coil. The existing prostate endorectal coils, the BPX-10 [1.0 Tesla], BPX-15 [1.5 Tesla], and BPX-30 [3.0 Tesla] can be used as a receive coil for imaging, and as a transmit coil for local RF heating of the prostate gland using RF energy at the Larmour frequency of the host MRI scanner. To accomplish this, the interface would essentially duplicate the function of the current interface devices such as the MEDRAD ATD-II in the receive mode for imaging. Further, the system body coil would be used for RF transmission during imaging. Optionally, the endorectal coil could transmit for imaging, although the transmit B1 RF field might be too non-uniform to make this a desirable practice.

The endorectal coil would operate as a transmit coil to achieve RF heating of the prostate gland. The RF power could be obtained from the Head Coil Port on earlier GEHC MR systems, or from the Surface Coil Port that contains a transmit port on later GEHC systems, all Siemens MR systems, and new Philips MR systems. The new interface device could use the endorectal coil as a transmit coil; at 3.0 Tesla, the endorectal coil is ready to operate in this mode if driven by balanced RF energy source having a characteristic output impedance to the endorectal coil of 200 ohms balanced [in the format of two 50 ohm unbalanced lines with a 180° difference in the phase of the RF drive power]. The endorectal coil is not capable of very high power operation as it is currently designed and built; however, the power handling capability is probably sufficient for the 4°-5° heating capability that is believed to be appropriate. At 1.5 or 1.0 Tesla, the endorectal coil impedance is considerably higher, on the order of 700-1000 ohms, and unbalanced. A simple matching network in the specialized interface can accomplish this. Whether the 1.0 or 1.5 Tesla endorectal coil can accept sufficient power to achieve the desired amount of heating is not yet known, since these coils are an unbalanced feed, use only one capacitor on the endorectal coil loop to series-resonate the loop, and represent a considerably higher impedance load. Potentially, 42 or 64 MHz energy may be less efficient at heating tissue than the 123-128 MHz RF energy employed at 3.0 Tesla. The fact that the 1.0 and 1.5 Tesla endorectal coils are Faraday shielded will not help the heating efficiency, either, since the Faraday shield greatly reduces the local electric field from the endorectal coil at 1.5 or 1.0 Tesla. A unique Coil Name and coil configuration file [or equivalent in non-GEHC systems] would allow the heating to be administered using the transmit/receive head coil RF power level to provide the heating energy; this can readily be done on GEHC and Siemens Medical Solutions systems, and should be possible on newer Philips systems that include at least one transmit RF port in the Surface Coil Port. Manipulation of the MRI system parameters [TR, scan type, flip angle, echo train length] can control the amount of RF heating that is generated,

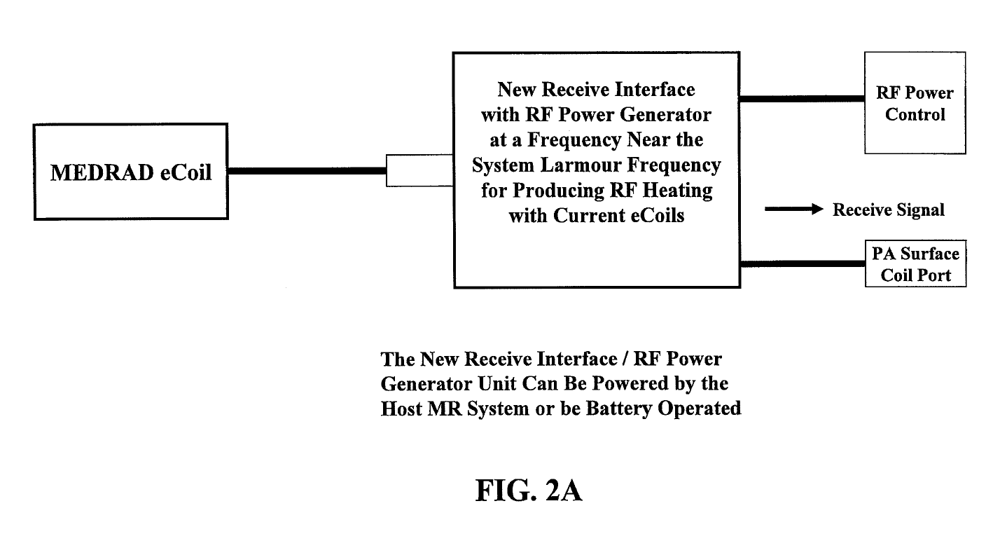

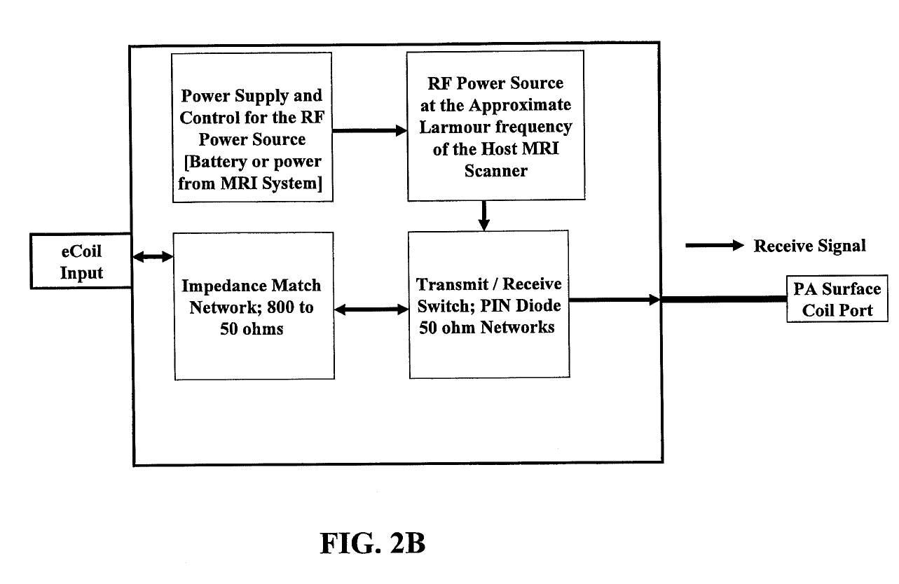

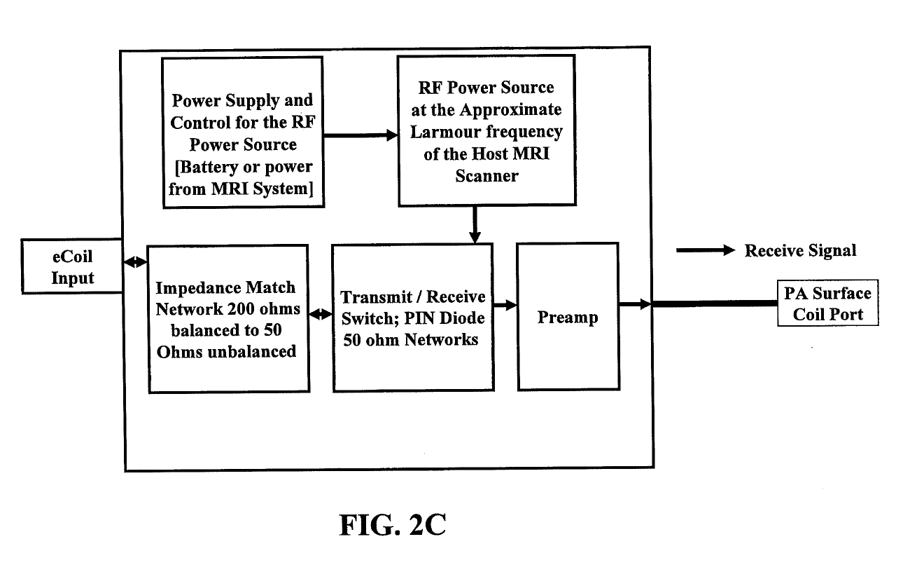

FIGS. 2A, 2B and 2C show Imaging and RF Heating with Existing endorectal coil Products and a Separate RF Source. The current endorectal coil products [MEDRAD BPX-30, BPX-15, and BPX-10] could also be used with a unique interface device that was both the receive-mode interface to the host MRI scanners from GEHC, Siemens, or Philips, and also an RF energy source at an appropriate frequency for the endorectal coil in use. In this format, the RF energy source would be operating at a frequency that was at least near the Larmour frequency of the host MRI system. This would allow the new interface device to control the RF heating energy in a very exact manner, independent of the MRI scanner in use. The RF energy source could be used in the MR scanner bore, or in the MR scanner shielded room while not inside of the MR system magnet bore. The specialized interface device could possibly operate outside of the MR shielded room or scanner environment, but issues with FCC Part 15 RF radiation levels might prohibit such operation; interference issues with other local MRI systems might also be an issue if operation outside of the MR scanner shielded room is contemplated. The interface system described here would connect the endorectal coil to the host MRI scanner for imaging in the usual way; for RF heating, the interface would provide a controlled amount of RF power [probably Continuous Wave, with variable power levels] to the endorectal coil for an appropriate and controlled period of time. The endorectal coil could again be used for imaging whenever desired [as long as the endorectal coil was not being actively used for RF transmission and heating at the exact time that imaging was desired].

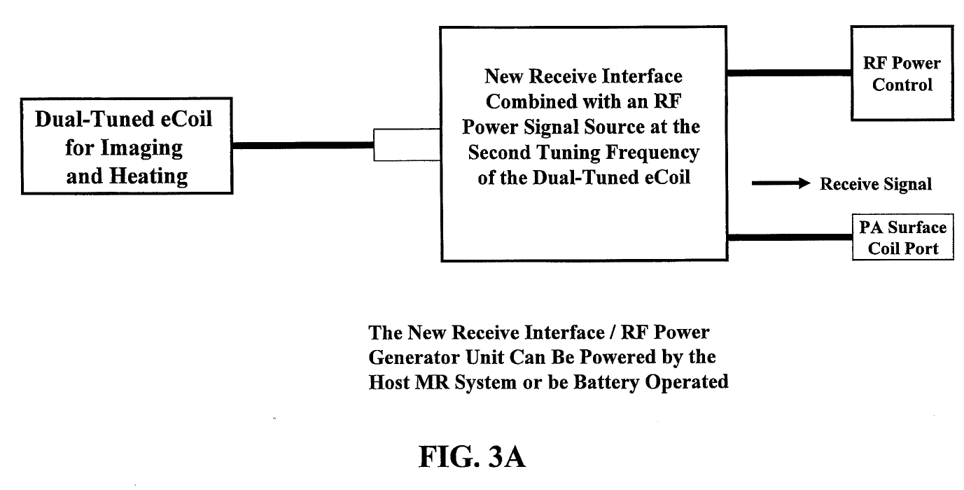

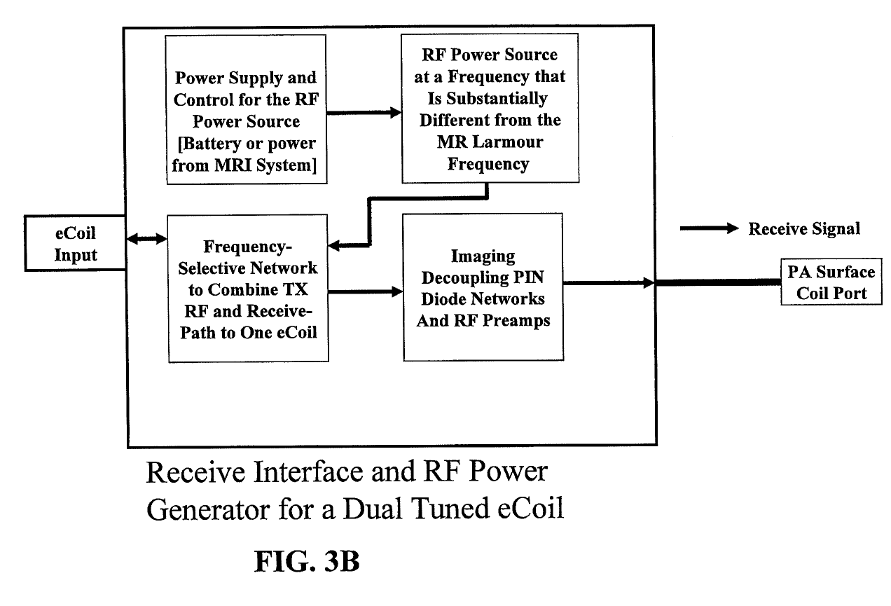

FIGS. 3A and 3B depict a New, Dual-Tuned endorectal coil for Imaging and RF Heating. With this configuration, the endorectal coil itself would be modified to continue to provide imaging performance as it currently does; the new endorectal coil could plug into the current interface devices sold by MEDRAD for imaging, and into a unique source of RF power for heating, or a combined interface system could be provided. The endorectal coil and RF source could operate at a frequency that is widely separated from the imaging frequency; for example, the heating RF source and the endorectal coil could operate at approximately 2450 MHz, a frequency that is the dipolar moment resonance frequency of water, and the frequency used by typical microwave ovens for the heating function, and at the MR system Larmour frequency for imaging.

The endorectal coils can be dual-tuned devices, or they could contain two separate coil systems, one for imaging, and one system for RF heating. The heating coil system could contain more than one coil element; this would allow some localization of the application of the heating. A dedicated RF source would be used with this system; the RF source could direct the RF energy to the appropriate transmit coil elements, and also control the RF power level and duration of application. There are a large number of possible variations in the hardware that would fall under this general category; the number of coil elements, the operating frequencies, and the nature of the RF power source all can rationally exist in a variety of forms.

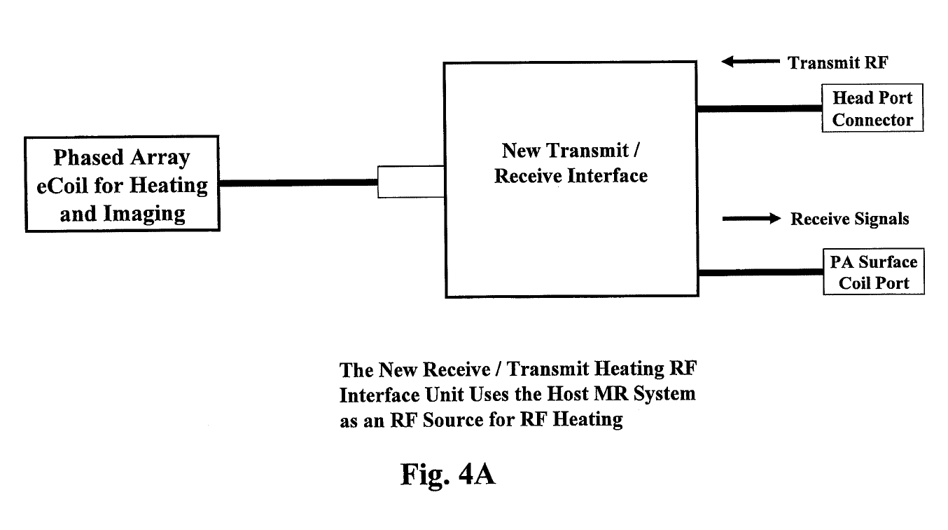

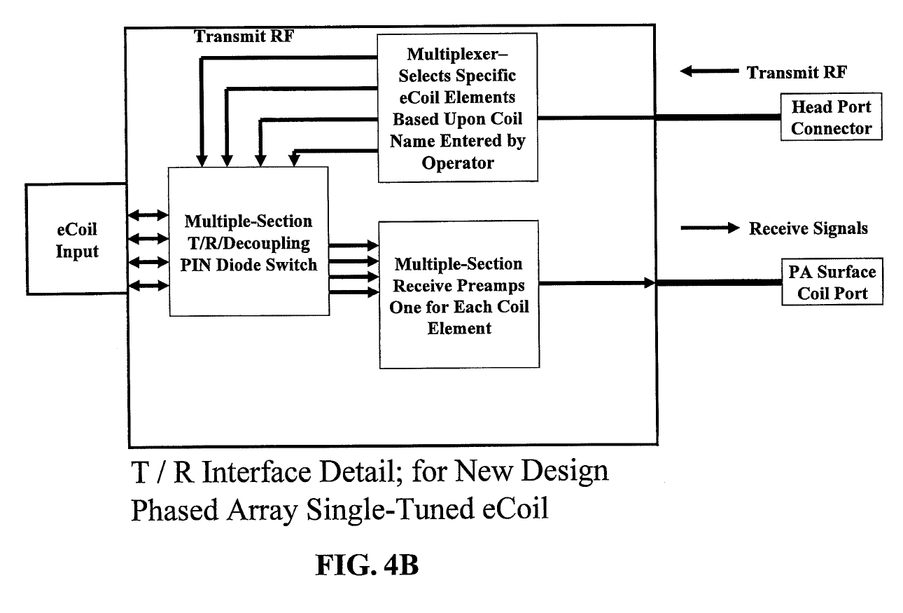

FIGS. 4A and 4B depict an Array Coil for Imaging and Localized RF Heating at the MR System Larmour Frequency. A new endorectal coil could be developed for use with a new interface for interfacing an array coil to the host MR system and for connecting RF power from the host MRI scanner to the desired coil elements to direct the RF heating to the desired areas. The purpose of this version of the new endorectal coil system is to localize the RF heating to some degree while maintaining all operation from the host MRI scanner system. The endorectal coil could contain from two to perhaps sixteen different elements; a coil with four to eight elements is probably a practical limit. This concept would not use a separate RF source; it would employ the host MRI scanner as the RF power source, and would apply the RF heating energy at the Larmour frequency of the MRI scanner.

The control of the endorectal coil transmit elements used could be accomplished, at least on GEHC systems and probably on Siemens and Philips systems, by using unique Coil Names with different Coil Configuration File parameters to control which elements are activated; the MR pulse sequence and duration selected could set the total amount of RF heating power delivered. The receive mode could simply use all of the endorectal coil elements, a selection of the endorectal coil elements, or a specific endorectal coil element under control of the Coil Name and the Configuration File selections in the standard phased array receive manner. An endorectal coil with this level of complexity may cease to be a disposable due to cost consideration; it could be designed as a long-life device, or a device with a limited but multiple-use life.

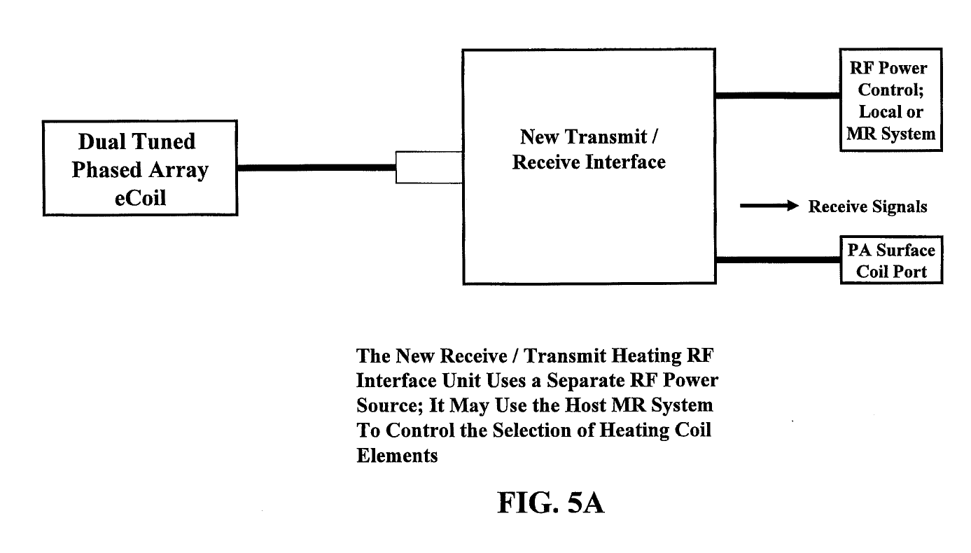

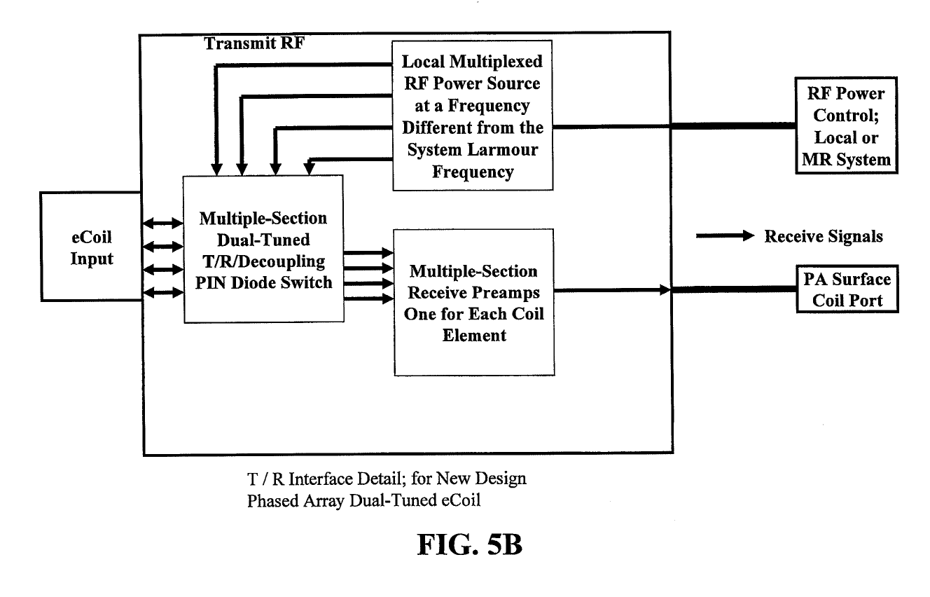

FIGS. 5A and 5B show a Dual-Tuned Phased Array Coil for Imaging and Localized RF Heating to Use a Frequency Different from the System Larmour Frequency. A new Phased Array Dual Tuned endorectal coil could be developed for imaging and RF heating: it would require a new interface with an RF power source at a frequency other than the system Larmour frequency. The coil element selection for the RF heating could be controlled by a selection of Coil Names on the host MRI scanner to direct the RF heating to the desired areas, or it could be controlled by other local or remote means. The purpose of this version of the new endorectal coil system is to localize the RF heating from an independent source of RF power, and to maintain imaging capability from the host MRI scanner system. The endorectal coil could contain from two to perhaps sixteen different elements; a coil with four to eight elements is probably a practical limit. This concept would use a separate RF source; it could optionally employ the host MRI scanner as the control device by selecting specific Coil Names to cause the independent RF power source to apply the RF energy selectively to the various transmit array elements in the endorectal coil. The RF heating energy would operate at some frequency that is different, perhaps substantially different, from the Larmour frequency of the MRI scanner.

The control of the endorectal coil transmit elements used could be accomplished, at least on GEHC systems and probably on Siemens and Philips systems, by using unique Coil Names with different Coil Configuration File parameters to control which elements are activated. The receive mode could simply use all of the endorectal coil elements in a standard phased array receive manner. For the Transmit RF Heating mode, a selection of the endorectal coil elements, or a specific endorectal coil element would be under control of the Coil Name and the Configuration File selections. Optionally, the interface device could contain its own local control for the selection of the coil elements to be used for heat application, or a remote control or wired or wireless design could be provided. An endorectal coil with this level of complexity may cease to be a disposable due to cost consideration; it could be designed as a long-life device, or a device with a limited but multiple-use life.

It is possible to employ the same basic concepts outlined in the five invention embodiments described above to Surface coil systems other than the intracavity or endorectal coils discussed above; this can be done in combination with endorectal coils or with external surface coil devices. In each case where the endorectal coil is described in a new application and/or new configuration, the endorectal coil device could be replaced with a specific surface coil system as might be appropriate for the anatomy of interest. Because the majority of current surface coil systems are not intended to be disposable devices, and are not used with an external interface device as is done with the MEDRAD family of endorectal coils, in most instances modifications will be needed to employ existing surface coils in a manner as described above. The applications and benefits outlined in this document can in many cases be implemented to benefit with current or newly developed surface coil systems. These surface coil systems could include an endorectal coil as a part of the system, or not include one as would be appropriate for the specific application and hardware.

The surface coil system, or specific elements of it, could operate as a transmit coil to achieve RF heating of the specific target anatomy. The RF power could be obtained from the Head Coil Port on earlier GEHC systems, or from the Surface Coil Port that contains a transmit port on later GEHC, all Siemens, and new Philips systems. A new interface device could use the modified surface coil system as a transmit coil. Current receive-only surface coils are, in general, not capable of very high power operation with minimal modifications; however, the power handling capability with simple modifications may be sufficient for the 3°-10° C. heating capability that is believed to be appropriate. Again, 42 or 64 MHz energy may be less efficient at heating tissue than the 123-128 MHz RF energy employed at 3.0 Tesla. A unique Coil Name and coil configuration file [or equivalent in non-GEHC systems] plus a modified surface coil and a specialized coil interface device would allow the heating to be administered using the transmit/receive head coil RF power level to provide the heating energy; this can readily be done on GEHC and Siemens Medical Solutions systems, and should be possible on newer Philips systems that include at least one transmit RF port in the Surface Coil Port. Manipulation of the MRI system parameters [TR, scan type, flip angle, echo train length] can control the amount of RF heating that is generated. A reference concerning operating a coil device or system as a receive-only coil or, selectively, as a transmit or transmit/receive coil is given in U.S. Pat. No. 6,512,374B1, the contents of which are incorporated herein by reference.

The present invention includes several novel and unique features. It employs RF energy from a locally applied antenna [the MR endorectal coil] to the prostate gland or colon. This disclosure includes several unique features that can, in general, be used independently or in combination with other features. In one embodiment, the invention includes a MEDRAD disposable endorectal MR coil antenna, the BPX-10, BPX-15, or BPX-30, and allows the endorectal coil to be used for MR imaging of the prostate; it also makes provision for the disposable endorectal coil to be used to apply RF energy to the prostate gland to cause RF heating of the area. The RF power source can be the host MRI scanner in an embodiment of this invention, or the Interface Device included as a part of this invention may contain its own RF power source. The energy to operate the RF power source if used can come from an external source, an internal battery, or from the host MR scanner via power harvested by any one of a number of different means. These means may include DC connections to the DC power sources of the host MR system supplied at the Surface Coil Port, or from bias currents provided by the host MRI system at the port, or from a harvesting of RF energy from the host MRI system. The endorectal coil may be designed to resonate at two different frequencies, one at the Larmour frequency of the host MRI scanner, and at a second frequency removed from the Larmour frequency. The second frequency might be about 2450 MHz, the dipolar frequency of water molecules, or some other desired frequency that is not the Larmour frequency of the MR scanner. The endorectal coil may be designed to be an array coil, in the transmit, receive, or both modes. The use of array technology could allow some focusing of the applied RF power to specific regions of the prostate gland or colon. The phased array aspect of the design can be used with a single-tuned coil system operating at the host MR system Larmour frequency, or it may be dual-tuned to allow the use of a frequency other than the system Larmour frequency, such as [but not limited to] 2450 MHz.

What is claimed is:

1. A method comprising:
creating an image of tissue with an MR coil;
creating a temperature contour map of the tissue; and
heating the tissue with the MR coil.

2. The method of claim 1 wherein the tissue is heated to a desired thermal profile.

3. The method of claim 1 wherein the MR coil is used to create the temperature contour map of the tissue.

4. A method comprising:
creating an image of tissue with an MR scanner; and
heating the tissue with the MR scanner.

5. A method comprising:
placing an MR coil in proximity to internal tissue of a patient;
creating an MR image of the internal tissue with the MR coil;
heating the internal tissue with the MR coil.

6. The method of claim 5 wherein the MR coil is an endorectal coil.

7. A system comprising:
an array of RF elements that can modify the deposition of energy in tissue by controlling the phase or frequency of the energy.

8. A system comprising:
an antenna array; and
a closed loop control system in communication with the antenna array, the closed loop control system operable to control the antenna array to match the thermal treatment plan for a patient to a measured temperature distribution in a target tissue.
